(19) United States
(12) Patent Application Publication
(10) Pub. No.: US 2005/0276847 A1
(43) Pub. Date: Dec. 15, 2005

(54) Oral delivery system

(76) Inventors: Michael Stephen Roberts, Queensland (AU); George Alexander Davidson, Queensland (AU)

Correspondence Address: KENYON & KENYON, 1500 K STREET NW, SUITE 700, WASHINGTON, DC, 20005, US

(21) Appl. No.: 11/138,262

(22) Filed: May 27, 2005

Related U.S. Application Data
(60) Provisional application No. 60/575,477, filed on May 28, 2004.

(51) Int. Cl. A61K 9/20; A61K 9/16

(52) U.S. Cl. 424/464

(57) ABSTRACT

The present invention relates generally to formulations comprising paracetamol. More particularly, the present invention provides a swallow formulation comprising paracetamol which facilitates the rapid delivery of paracetamol into the circulatory system following oral administration. The present invention further relates to methods for inducing efficient pain relief including an analgesic effect by the administration of the paracetamol formulation.

### CROSS REFERENCE TO RELATED APPLICATIONS

This application claims the benefit of U.S. provisional application Ser. No. 60/575,477, filed May 28, 2004, the contents of which are incorporated herein in their entirety.

### FIELD OF THE INVENTION

The present invention relates generally to formulations comprising paracetamol. More particularly, the present invention provides a swallow formulation comprising paracetamol which facilitates the rapid delivery of paracetamol into the circulatory system following oral administration. The present invention further relates to methods for inducing efficient pain relief including an analgesic effect by the administration of the paracetamol formulation.

### BACKGROUND OF THE INVENTION

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Paracetamol, also known as N-acetyl-p-aminophenol, acetaminophen and APAP, is an analgesic and antipyretic agent and is widely used in prescription and non-prescription medicines. Paracetanol was first marketed in the 1950's and is now a commonly used agent (Prescott Am. J. Ther. 7(2):143-147 2000). The precise mechanism of paracetamol's analgesic and antipyretic effect remains unclear. However, some studies have suggested that the rate of administration is a factor (Nielsen et al. Eur. J. Clin. Pharmacol. 42(3): 261-264, 1992, Luthy, et al. Schweiz Med. Wochenschr 123 (Suppl 50)/II:406, 1993). Accordingly, increasing the rate of absorption of paracetamol should enable a greater and more rapid analgesic effect after oral dosing. In this regard, oral delivery is the most convenient and acceptable route of drug administration to end users, especially for a drug administered at high doses and frequency, such as paracetamol.

Improving the rate and extent of absorption of oral formulations of compounds has been the subject of substantial research. In general, once a solid swallow composition reaches the stomach, it undergoes disintegration and/or dissolution and passes into the small intestine where the active ingredient is absorbed across intestinal walls into the circulatory system via the portal vein and liver before reaching the site of action.

Rates of absorption are often assessed by comparing standard pharmacokinetic parameters such as the time to peak plasma concentration (Tₘₐₓ) and the peak plasma concentration (Cₘₐₓ). The extent of absorption is assessed by the area under the plasma concentration-time curve (AUC). A short Tₘₐₓ has been used to indicate rapid absorption. The FDA Guidance for Industry on Bioavailability and Bioequivalence Studies for Orally Administered Products (2003) and related publications (Chen et al, Clin. Pharmacokinet. 40(8):565-72, 2001) recommend the use of partial AUC as an early exposure measure, such that a high partial AUC will be an indicator of rapid absorption. The parameters for known formulations vary greatly between subjects. The parameters also vary depending on aspects of the study protocol such as the sampling scheduling, subject posture and general subject health. Values quoted in this specification are given as mean±standard deviation unless otherwise noted.

For paracetamol tablets supplied by McNeil, a Tₘₐₓ value has been quoted as 45.6 minutes with a standard error of 7.2 minutes with a corresponding Cₘₐₓ value of 11.99 mg.L⁻¹ with a standard error of 1.02 (Ameer et al., J. Pharm. Sci, 72:955-958, 1983)). Other values quoted include a Tₘₐₓ of 35.6±27.7 minutes and Cₘₐₓ of 9.47±4.18 mg.L⁻¹ (Rumble et al., Clin. Pharmacokinet. 20 (2):167-173, 1991), a Tₘₐₓ of 1.82 hours with a standard error of 0.46 hours and a Cₘₐₓ of 20.4±3.2 mg.L⁻¹ for Paralen tablets and for Panadol tablets, a Tₘₐₓ of 35 minutes and as high as 77 minutes and a Cₘₐₓ value of 17.02±6.04 mg.L⁻¹ (Grattan et al., Eur. J. Pharm. Biopharm. 49(3):225-229, 2000).

The range of paracetamol plasma concentrations for effective analgesia in humans is quoted to be about 5-20 μg.mL⁻¹. (Prescott, Medical Clinics of North America, 58:907-916, 1974). For antipyresis a paracetamol concentration of 10-20 μg.mL⁻¹ has been shown to be effective (Rumack et al, Pediatrics 62(Suppl):898-903, 1978).

In a submission compiled by McNeil in 2002 in relation to Tylenol to demonstrate safety and efficacy of paracetamol to the United States Food and Drug Administration (USFDA), the effective concentration which elicits 50% of the maximum drug response (EC₅₀) was estimated to be between 15.2 μg.mL⁻¹ and 16.55 μg.mL⁻¹ (McNeil submission to FDA, 2002)

Prescott (1974, supra) reported there may be as much as an 80-fold range in concentrations 1 hour after therapeutic doses of paracetamol were administered in 43 patients. He concluded that pharmacokinetic variability impacts on response rates for analgesics. In fact, many patients may never achieve a therapeutic effect.

In U.S. Pat. No. 6,316,025, Grattan describes a swallow tablet of paracetamol containing 300 mg to 1000 mg of sodium bicarbonate per tablet and a paracetamol to sodium bicarbonate ratio of between 0.74 and 1. Grattan et al. (2000 supra) subsequently reported that a formulation with 630 mg sodium bicarbonate gave a Tₘₐₓ of 17.5±4.95 minutes and a Cₘₐₓ of 29.79±9.06 mg.L⁻¹. It was suggested that this was due to an osmotic effect of sodium bicarbonate, which would be isotonic when ingested with 100 mL of water.

U.S. Patent Application No. 20040204475 describes a formulation containing sodium bicarbonate and eletriptan. The sodium bicarbonate is administered in an amount to obtain a duodenal concentration approximately isotonic with serum (150 mmol). The formulations exemplified all contained 630 mg sodium bicarbonate.

U.S. Patent Application No. 20040170681 describes a paracetamol formulation containing less than 100 mg sodium bicarbonate per tablet. About 90% of the paracetamol is described as being released from this formulation in 15 minutes using United States Pharmacopoeia (USP) dissolution apparatus 2 with 900 mL 0.05 N hydrochloric acid at 30 rpm and 37° C. A formulation was exemplified which resulted in an area under the plasma concentration-time curve at 20 minutes after administration (AUC20) of 17 μg.min.mL⁻¹ in fed subjects when given as a 1000 mg paracetamol dose.

United Kingdom Patent No. 2103087 describes the use of antacids in paracetamol tablets. Antacids including calcium carbonate and sodium bicarbonate were used in the range of 60-1200 mg. The greatest increase was noted with 225 mg of sodium bicarbonate which showed an increase in the rate of absorption of paracetamol of between 7% and 31% compared with conventional paracetamol tablets. The shortest Tₘₐₓ reported was 29 minutes.

In accordance with the present invention, paracetamol formulations and in particular swallow formulations are defined in which the parameters for drug dissolution and absorption have been significantly improved.

### SUMMARY OF THE INVENTION

The present invention relates generally to paracetamol formulations in the form of fast absorbing oral delivery systems. In particular, the present invention provides a swallow formulation comprising paracetamol, one or more pH modulating agents, and one or more agents which facilitate water uptake. The paracetamol is incorporated as a rapidly dissolving form of paracetamol.

The present invention provides, therefore, a swallow formulation comprising a rapidly dissolving form of paracetamol, a pH modulating agent and an agent which facilitates water uptake, wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, and wherein at least about 70% of the paracetamol in the swallow formulation is dissolved from the swallow formulation within 180 seconds in USP dissolution apparatus 2 with 900 mL 0.05 N hydrochloric acid at 30 rpm and 37° C.

A rapidly dissolving form of paracetamol includes paracetamol having a volume median diameter (D₅₀) of less than 350 μm and a surface area of greater than 0.07 m².g⁻¹. It also includes any paracetamol preparation which exhibits a dissolution rate in USP dissolution apparatus 2 using 900 mL of 0.05 N hydrochloric acid at 30 rpm and 37° C. of at least 30% in 180 seconds.

Accordingly, another aspect of the present invention is directed to a swallow formulation comprising a form of paracetamol having a volume median diameter (D₅₀) of less than 350 μm and a surface area of greater than 0.07 m².g⁻¹; a pH modulating agent in an amount sufficient to neutralize from about 0.6 mL to about 110 mL 0.1 N hydrochloric acid and/or to neutralize from about 0.06 mmol to about 11 mmol of a monoprotic acid; and an agent which facilitates water uptake; wherein at least 70% of the paracetamol is dissolved from the swallow formulation within 180 seconds in USP dissolution apparatus 2 with 900 mL 0.05 N hydrochloric acid at 30 rpm and 37° C.

In another embodiment, the present invention contemplates a swallow formulation comprising a form of paracetamol which exhibits a dissolution rate in USP dissolution apparatus 2 using 900 mL of 0.05 N hydrochloric acid at 30 rpm and 37° C. of at least 30% in 180 seconds; a pH modulating agent in an amount sufficient to neutralize from about 0.6 mL to about 110 mL 0.1 N hydrochloric acid and/or to neutralize from about 0.06 mmol to about 11 mmol of 0.1 N of a monoprotic acid; and an agent which facilitates water uptake; wherein at least 70% of the paracetamol is dissolved from the swallow formulation within 180 seconds in USP dissolution apparatus 2 with 900 mL 0.05 N hydrochloric acid at 30 rpm and 37° C.

The swallow formulation of the present invention achieves on administration of 1000 mg paracetamol, a mean AUC20 (i.e. area under the plasma concentration-time curve at 20 minutes after administration) of more than 150 min.mg.L⁻¹ in fasted healthy human subjects.

Accordingly, another aspect of the present invention provides a swallow formulation comprising a form of paracetamol having a volume median diameter (D₅₀) of less than 350 μm and a surface area of greater than 0.07 m².g⁻¹; a pH modulating agent in an amount sufficient to neutralize from about 0.6 mL to about 110 mL 0.1 N hydrochloric acid and/or to neutralize from about 0.06 mmol to about 11 mmol of a monoprotic acid; and an agent which facilitates water uptake; wherein an administration of 1000 mg paracetamol achieves a mean AUC20 of more than 150 min.mg.L⁻¹ in fasted healthy human subjects.

In a related aspect, the present invention provides a swallow formulation comprising a form of paracetamol which exhibits a dissolution rate in USP dissolution apparatus 2 using 900 mL of 0.05 N hydrochloric acid at 30 rpm and 37° C. of at least 30% in 180 seconds; a pH modulating agent in an amount sufficient to neutralize from about 0.6 mL to about 110 mL 0.1 N hydrochloric acid and/or to neutralize from about 0.06 mmol to about 11 mmol of a monoprotic acid; and an agent which facilitates water uptake; wherein an administration of 1000 mg paracetamol achieves a mean AUC20 of more than 150 min.mg.L⁻¹ in fasted healthy human subjects.

Another aspect of the invention provides a dosage form such as a coated tablet, uncoated tablet, capsule, powder, paste, cachet, colloid, gel or melt.

The present invention further contemplates a method for treating therapeutic indications including an analgesic or antipyretic effect in a human subject said method comprising administering to said subject a pain relieving effective amount of a swallow formulation comprising a rapidly dissolving form of paracetamol having a volume median diameter (D₅₀) of less than 350 μm and a surface area of greater than 0.07 m².g⁻¹ with a pH modulating agent and an agent which facilitates water uptake into to the swallow formulation, wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, and wherein at least about 70% of the paracetamol in the swallow formulation is dissolved from the swallow formulation within 180 seconds in USP dissolution apparatus 2 with 900 mL 0.05 N hydrochloric acid at 30 rpm and 37° C.

The present invention is further directed to the use of a form of paracetamol which achieves on administration of 1000 mg paracetamol, a mean AUC20 of more than 150 min.mg.L⁻¹ in fasted healthy human subjects in the manufacture of a medicament for the inducement of pain relief in a human subject.

In another embodiment, the present invention contemplates a method for treating pain or fever in a human subject said method comprising administering to said subject a pain relieving or fever reducing effective amount of a swallow formulation comprising a rapidly dissolving form of paracetamol having a volume median diameter (D₅₀) of less than 350 μm and a surface area of greater than 0.07 m².g⁻¹ with a pH modulating agent and an agent which facilitates water uptake into to the swallow formulation, wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, and wherein an administration of 1000 mg paracetamol achieves a mean AUC20 of more than 150 min.mg.L¹ in fasted healthy human subjects.

In still another embodiment, the present invention provides a method for inducing pain relief including an analgesic effect in a human subject said method comprising administering to said subject a pain relieving effective amount of a swallow formulation comprising a rapidly dissolving form of paracetamol which exhibits a dissolution rate in USP dissolution apparatus 2 using 900 mL of 0.05 N hydrochloric acid at 30 rpm and 37° C. of at least 30% in 180 seconds with a pH modulating agent and an agent which facilitates water uptake into to the tablet, wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, and wherein at least about 70% of the paracetamol in the swallow formulation is dissolved from the swallow formulation within 180 seconds in USP dissolution apparatus 2 with 900 mL 0.05 N hydrochloric acid at 30 rpm and 37° C.

### DETAILED DESCRIPTION OF THE INVENTION

The present invention provides a paracetamol formulation and in particular a rapid dissolving formulation form of paracetamol. The formulation is generally referred to herein as a swallow formulation. The swallow formulation generally comprises rapidly dissolving paracetamol combined with a pH modulating agent and an agent which facilitates uptake of water. The formulation of the present invention may optionally be administered with water or any other aqueous-based fluid.

The present invention provides, therefore, a swallow formulation comprising a rapidly dissolving form of paracetamol, a pH modulating agent and an agent which facilitates water uptake, wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, and wherein at least about 70% of the paracetamol in the swallow formulation is dissolved from the swallow formulation within 180 seconds in USP dissolution apparatus 2 with 900 mL 0.05 N hydrochloric acid at 30 rpm and 37° C.

It is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to “an agent” includes a single agent, as well as two or more agents; reference to “a pH modulating agent” includes a single pH modulating agent, as well as two or more pH modulating agents, reference to “a water uptake agent” includes a single water uptake agent or two or more water uptake agents; and so forth.

The rapidly dissolving form of paracetamol includes a paracetamol having a volume median diameter (D₅₀) of less than 350 μm and a surface area of greater than 0.07 m².g⁻¹. Alternatively, or in addition, it includes any paracetamol preparation which exhibits a dissolution rate in USP dissolution apparatus 2 using 900 mL of 0.05 N hydrochloric acid at 30 rpm and 37° C. of at least 30% in 180 seconds.

Accordingly, another aspect of the present invention provides a swallow formulation comprising a form of paracetamol having a volume median diameter (D₅₀) of less than 350 μm and a surface area of greater than 0.07 m².g⁻¹; a pH modulating agent in an amount sufficient to neutralize from about 0.6 mL to about 10 mL 0.1 N hydrochloric acid and/or to neutralize from about 0.06 mmol to about 11 mmol of a monoprotic acid; and an agent which facilitates water uptake; wherein at least 70% of the paracetamol is dissolved from the swallow formulation within 180 seconds in USP dissolution apparatus 2 with 900 mL 0.05 N hydrochloric acid at 30 rpm and 37° C.

In a related aspect, the present invention is directed to a swallow formulation comprising a form of paracetamol which exhibits a dissolution rate in USP dissolution apparatus 2 using 900 mL of 0.05 N hydrochloric acid at 30 rpm at 37° C. of at least 30% in 180 seconds; a pH modulating agent in an amount sufficient to neutralize from about 0.6 mL to about 110 mL 0.1 N hydrochloric acid and/or to neutralize from about 0.06 mmol to about 11 mmol of a monoprotic acid; and an agent which facilitates water uptake, wherein at least 70% of the paracetamol is dissolved from the swallow formulation within 180 seconds in USP dissolution apparatus 2 with 900 mL 0.05 N hydrochloric acid at 30 rpm and 37° C.

The swallow formulation of the present invention achieves on administration of 1000 mg paracetamol a mean AUC 20 of more than 150 min.mg.L⁻¹ in fasted healthy human subjects.

Accordingly, another aspect of the present invention provides a swallow formulation comprising a form of paracetamol having a volume median diameter (D₅₀) of less than 350 μm and a surface area of greater than 0.07 m².g⁻¹; a pH modulating agent in an amount sufficient to neutralize from about 0.6 mL to about 110 mL 0.1 N hydrochloric acid and/or to neutralize from about 0.06 mmol to about 11 mmol of a monoprotic acid; and an agent which facilitates water uptake; wherein an administration of 1000 mg paracetamol achieves a mean AUC20 of more than 150 min.mg.L¹ in fasted healthy human subjects.

In a related aspect, the present invention provides a swallow formulation comprising a form of paracetamol which exhibits a dissolution rate in USP dissolution apparatus 2 using 900 mL of 0.05 N hydrochloric acid at 30 rpm and 37° C. of at least 30% in 180 seconds; a pH modulating agent in an amount sufficient to neutralize from about 0.6 mL to about 110 mL 0.1 N hydrochloric acid and/or to neutralize from about 0.06 mmol to about 11 mmol of a monoprotic acid; and an agent which facilitates water uptake wherein an administration of 1000 mg paracetamol results in a mean AUC20 of more than 150 min.mg.L⁻¹ in fasted healthy human subjects.

In describing and claiming the present invention, the following terminology is used in accordance with the definitions set forth below.

The terms “active agent”, “compound”, “pharmacologically active agent”, “medicament”, “active”, “active ingredient”, “drug” and “drug component” are used interchangeably throughout this specification. The terms also encompass pharmaceutically acceptable and pharmacologically active ingredients of those active agents specifically mentioned herein including but not limited to salts, esters, amides, pro-drugs, active metabolites, analogs and the like. When the terms “active agent”, “compound”, “pharmacologically active agent”, “medicament”, “active”, “drug”, “drug component” and “paracetamol” are used, then it is to be understood that this includes those compounds per se as well as pharmaceutically acceptable, pharmacologically active salts, esters, amides, pro-drugs, metabolites, analogs, etc. The terms “agent”, “compound” etc may be a single molecule or a composite of molecules.

By the term “effective amount” or “therapeutically effective amount” of paracetamol as used herein means that a sufficient amount of paracetamol is used to provide the desired therapeutic effect or the desired pharmacological, physiological or biochemical event including the amelioration of symptoms being treated or prevention of symptoms. Of course, undesirable effects, e.g. side effects, are sometimes manifested along with the desired therapeutic effect; hence, a practitioner balances the potential benefits against the potential risks in determining what is an appropriate “effective amount”.

The terms “delivery” and “administration” are used interchangeably throughout the specification to mean the act of providing the oral dosage form to an individual. The term “administering” is considered herein synonymous with “delivering”, “providing”, “introducing” or “swallowing”.

By “pharmaceutically acceptable excipient” is meant a pharmaceutical vehicle comprised of a material that is not biologically or otherwise undesirable, i.e. the oral dosage form may be administered to a subject along with paracetamol without causing any or a substantial adverse reaction. Excipients may include carriers and other additives such as diluents, binders, detergents, coloring agents, flavoring agents, wetting or emulsifying agents, preservatives, glidants, lubricants and the like as well as disintegrants.

Similarly, a “pharmacologically acceptable” salt, ester, amide, pro-drug or derivative of paracetamol as provided herein is a salt, ester, amide, pro-drug or derivative that is not biologically or otherwise undesirable.

The terms “treating” and “treatment” as used herein refer to reduction or amelioration in severity and/or frequency of symptoms, elimination of symptoms and/or underlying cause and/or prevention of the occurrence of symptoms and/or their underlying cause. Thus, for example, “treating” a subject involves prevention of a particular disorder or adverse physiological event in a susceptible individual as well as treatment of a clinically symptomatic individual by inhibiting or causing regression of a particular condition. Thus, for example, a method of treating a subject in need of pain relief encompasses both prevention of pain as well as treating conditions of pain. Reference to the treatment of pain includes the induction of analgesia. In addition, the subject formulation is useful for treating the symptoms of conditions requiring pain relief.

Conditions contemplated herein include all conditions responsive to paracetamol including, but not limited to, pain and fever management including pain and/or fever relief, pain and/or fever prevention, pain and/or fever reduction and/or treatment of levels of pain and/or fever.

Cₘₐₓ is the peak paracetamol plasma concentration. AUC20 is the partial area under the plasma concentration-time curve for 20 minutes after administration. Tₘₐₓ is the time to reach peak paracetamol plasma concentration.

Reference to “a pH modulating agent” includes one or more than one pH modulating agents that will change the pH of an aqueous solution. These may include acids, bases or a combination of one or more acids and/or bases. Reference to more than one includes from 2 to about 10 such as 2, 3, 4, 5, 6, 7, 8, 9 or 10 pH modulating agents.

In one particular embodiment, at least one of the pH modulating agents is soluble and/or dispersible.

In another particular embodiment, at least one of the pH modulating agents is a base.

Non-limiting examples of suitable pH modulating agents include sodium carbonate, ammonium carbonate, sodium bicarbonate, potassium bicarbonate, calcium carbonate, magnesium carbonate, disodium glycine carbonate, sodium glycine carbonate, lysine carbonate, arginine carbonate, citric acid, tartaric acid, malic acid, fumaric acid, metatartaric acid, adipic acid, sodium acid citrate, ascorbic acid and amino acids such as aspartic acid, glutamic acid, glycine, leucine, tyrosine, tryptophan, glycine hydrochloride, glycine fumarate, and monophosphate glycine as well as combinations of two or more of the above.

Suitably, at least one of the pH modulating agents is a carbonate such as an alkaline metal carbonate.

Desirably, the carbonate is water soluble.

Preferably the pH modulating agent in the swallow formulation is capable of neutralizing between 12 and 95 mL of 0.1 N HCl. In addition, the pH modulating agent of the swallow formulation is capable of neutralizing between 1.2 and 9.5 mmol of a monoprotic acid.

The pH modulating agent is generally present in an amount form about 2% to about 90% by weight of swallow formulation. More preferably the pH modulating agent is present in an amount from about 2% to about 80%, and most preferably from about 2% to about 70% by weight of swallow formulation. Examples of percentage amounts by weight of pH modulating agent include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90%.

Preferably, the ratio of paracetamol to pH modulating agent is between 0.5:1 and 30:1 inclusive by weight. More preferably, the ratio of paracetamol to pH modulating agent is between 1:1 and 20:1 by weight; examples including 0.5:1, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 20:1, 21:1, 22:1, 23:1, 24:1, 25:1, 26:1, 27:1, 28:1, 29:1 or 30:1 by weight.

In one embodiment, the pH modulating agent is sodium bicarbonate and/or potassium bicarbonate and/or magnesium carbonate and/or citric acid and is present in an amount from about 2% to 75% by weight of the swallow formulation.

Paracetamol is conveniently present in an amount ranging from about 100 to 2000 mg, in one embodiment about 200 to 1000 mg, for example approximately 500 mg per formulation (e.g. tablet) and the pH modulating agent is present in an amount from 50 mg to 450 mg per 500 mg of paracetamol such as 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449 or 450 mg per 500 mg.

Preferably, the pH modulating agent is present in an amount from 100 to 300 mg per 500 mg of paracetamol in the swallow formulation. Even more preferably the pH modulating agent is sodium bicarbonate.

More preferably the administration of 1000 mg paracetamol in the swallow formulation with water to fasted healthy human subjects provides a mean AUC20 of more than about 170 min.mg.L⁻¹ in healthy fasted human subjects.

In one embodiment, the swallow formulation is a tablet and at least 70% of the paracetamol is dissolved from the swallow formulation within 180 seconds in USP dissolution apparatus 2 with 900 mL 0.05 N hydrochloric acid at 30 rpm and 37° C. Even more preferably, at least 80% of the paracetamol is dissolved in 180 seconds.

In another embodiment, the swallow formulation is a tablet and at least 70% of the paracetamol is dissolved from the swallow formulation within 120 seconds in USP dissolution apparatus 2 with 900 mL 0.05 N hydrochloric acid at 30 rpm and 37° C. Even more preferably, at least 80% of the paracetamol is dissolved in 120 seconds.

In yet another embodiment, the swallow formulation is a tablet and at least 70% of the paracetamol is dissolved from the swallow formulation within 90 seconds in USP dissolution apparatus 2 with 900 mL 0.05 N hydrochloric acid at 30 rpm and 37° C. Even more preferably, at least 80% of the paracetamol is dissolved in 90 seconds.

Reference to “paracetamol incorporated in the swallow formulation as a rapidly dissolving form of paracetamol” includes rapidly dissolving forms of paracetamol and any paracetamol which is converted to a rapidly dissolving form of paracetamol during the manufacturing process.

Rapidly dissolving forms of paracetamol include paracetamol having a surface area to mass ratio greater than about 0.08 m².g⁻¹ as measured by gas adsorption and/or paracetamol having a volume median diameter (D₅₀) particle size less than about 300 μm as measured by laser diffraction.

In one embodiment, the paracetamol is in a rapidly dissolving form wherein desirably the dissolution rate in USP dissolution apparatus 2 using 900 mL of 0.05 N HCL with the paddle spinning at 30 rpm at 37° C. is at least about 30% in 180 seconds.

Alternatively a standard form of paracetamol may be converted to a rapidly dissolving form during the manufacturing process wherein the finished product will meet a dissolution rate in USP dissolution apparatus 2 using 900 mL of 0.05 N HCL at 30 rpm and 37° C. of at least about 70% in 180 seconds.

In one preferred embodiment, the amount of paracetamol dissolved from the swallow formulation in the presence of a carbonate pH modulating agent is at least 2 times, preferably 3 time, and more preferably 5 times greater than the amount of paracetamol dissolved from a swallow formulation without a carbonate pH modulating agent after 30 seconds in a USP dissolution apparatus 2 with 900 mL 0.05N hydrochloric acid at 30 rpm and 37° C.

In one embodiment, the rapidly dissolving paracetamol has a D₅₀ particle size less than 300 μm.

Rapidly dissolving forms of paracetamol include micro- or sub-micron particles and modified crystals of paracetamol as well as particles having a reduced particle size and/or increased surface area.

In a further embodiment, the rapidly dissolving form of paracetamol is a salt, ester, amide, pro-drug or other pharmaceutically acceptable derivative of paracetamol.

In one embodiment, paracetamol crystals are re-crystallised in the presence of a crystallization modifier such as a polymer or protein or mixtures thereof to produce modified crystals. Polymers which may be used include polyvinlypyrrolidone (PVP) and copolymers with polyvinlypyrrolidone subunits. Proteins which may be used include albumin (bovine or ovine), papain, pepsin and others. Preferably, polyvinlypyrrolidone is present during crystallization. Methods of re-crystallizing paracetamol in the presence of a crystallization modifier are well known to those of ordinary skill in the art.

Preferably the PVP—paracetamol co-crystal has a surface area to mass ratio of greater than 0.2 m².g⁻¹ as measured by gas adsorption.

Conveniently, the modified paracetamol crystals have a D₅₀ particle size in the range of 50-300 μm such as 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 18-1, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299 or 300 μm.

In one embodiment, the rapidly dissolving form of paracetamol is in the form of granules manufactured by granulation techniques including wet massing, dry slugging or compaction, fluidized bed or spray drying and may include the use of high energy granulators. The granules may also contain a pH modulating agent and/or a water uptake agent. The swallow formulation may optionally contain extra granular paracetamol and/or a pH modulating agent and/or water uptake agent.

Preferably, the granules include one or more disintegrants, such as but not limited to crospovidone, croscarmellose, sodium starch glycolate, starch and/or starch derivatives.

In one embodiment, the rapidly dissolving form of paracetamol is provided in a formulation comprising further molecules which enhance the dissolution of the paracetamol, such as eutectics including mannitol.

Accordingly, another embodiment of the present invention is directed to a swallow formulation comprising paracetamol incorporated as a rapidly dissolving form of paracetamol as described herein and a pH modulating agent wherein the pH modulating agent is in an amount sufficient to neutralize from 0.6 mL to 110 mL 0.1 N hydrochloric acid and which permits at least about 70% of the paracetamol to dissolve from the swallow formulation within 180 seconds in USP dissolution apparatus 2 with 900 mL 0.05 N hydrochloric acid at 30 rpm and 37° C. and which achieves on administration of a swallow formulation totaling 1000 mg paracetamol a mean AUC20 of more than 150 min.mg.L⁻¹ in fasted healthy human subjects.

The skilled addressee will appreciate, the swallow formulation may also comprise one or more pharmaceutically acceptable excipients.

Reference to “a water uptake agent” includes any agent which will facilitate the uptake of water by absorbing, dissolving, or wicking water. These may include wicking agents, disintegrants, binders, carriers and other hydrophilic excipients. Generally, but not exclusively, a water-uptake agent facilitates uptake of water into the swallow formulation. One of ordinary skill in the art would readily recognize agents that are water uptake agents.

Suitable water uptake agents include cross-linked polyvinylpyrrolidone (crospovidone), croscarmellose sodium, sodium starch glycolate, starch, starch derivatives, low substituted hydroxypropylcellulose, alginic acid, sodium alginate, calcium sulfate, calcium carboxymethylcellulose, microcrystalline cellulose, powdered cellulose, colloidal silicon dioxide, docusate sodium, guar gum, hydroxypropyl cellulose, magnesium aluminium silicate, methylcellulose, polacrilin potassium, silicified microcrystalline cellulose, magnesium oxide, tragacanth, mannitol, sorbitol, xylitol, sucrose, lactose, fructose, maltose, polyethylene glycol, aminoacids, cyclodextrin, urea and/or polyvinylpyrrolidone (povidone, PVP).

The water uptake agent may be present in an amount from 2% to 80% by weight of the swallow formulation and more preferably between 2% and 60% by weight of the swallow formulation.

Preferably, the ratio of water uptake agent to pH modulating agent is between 0.1:1 and 20:1 by weight such as 0.1:1, 0.2:1, 0.3:1, 0.4:1, 0.5:1, 0.6:1, 0.7:1, 0.8:1, 0.8:1, 0.9:1, 1:1, 2:1, 3:1, 4:1, 5:1, 6:1, 7:1, 8:1, 9:1, 10:1, 11:1, 12:1, 13:1, 14:1 or 15:1, 16:1, 17:1, 18:1; 19:1 and 20:1. More preferably the ratio of water uptake agent to pH modulating agent is between 0.3:1 and 15:1 by weight.

Optionally, the swallow formulation may also comprise one or more pharmaceutically acceptable excipients or other components such as flavoring agents, coloring agents, sweeteners and preservatives.

Accordingly, another aspect of the present invention is directed to a swallow formulation comprising paracetamol incorporated as a rapidly dissolving form of paracetamol as described herein, one or more pH modulating agents wherein the pH modulating agent is in an amount sufficient to neutralize from about 0.6 mL to about 110 mL 0.1 N hydrochloric acid and a water uptake agent in an amount up to 80% by weight of the swallow formulation which permits at least about 70% of the paracetamol to dissolve from the swallow formulation within 180 seconds in USP dissolution apparatus 2 with 900 mL 0.05N hydrochloric acid at 30 rpm and 37° C. and which achieves on administration of a swallow formulation totaling 1000 mg paracetamol a mean AUC20 of more than 150 min.mg.L⁻¹ in fasted healthy human subjects.

In one embodiment, the swallow formulation is co-administered with an aqueous fluid such as water. The co-administered fluid may be administered, before, after or with the swallow formulation.

Accordingly, another aspect of the present invention is directed to a swallow formulation comprising paracetamol incorporated as a rapidly dissolving form of paracetamol as described herein, a water uptake agent and one or more pH modulating agents wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, which permits at least about 70% of the paracetamol to dissolve from the swallow formulation within 180 seconds in USP dissolution apparatus 2 with 900 mL 0.05N hydrochloric acid at 30 rpm and 37° C. said dosage form further comprising one or more pharmaceutically acceptable carriers, diluents and/or excipients, wherein the swallow formulation is co-administered with fluid.

The paracetamol or salt or pro-active form may be provided from about 100 mg to about 1000 mg per swallow formulation such as 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999 or 1000 mg or a fraction in between. A range of from about 400 to 750 mg is particularly preferred such as from about 450 mg to about 700 mg such as 500 mg or 650 mg per swallow formulation.

The skilled addressee will appreciate the preferred amount of paracetamol will depend upon the intended user of the swallow formulation. For example, in infants under two months of age with post immunization fever 10 to 15 mg paracetamol per kilogram is the recommended dosage. For an adult dose of 1000 mg paracetamol the dose may be administered as a single dose administration comprising one, two, three, four or more swallow formulations. For example, a 1000 mg dose of paracetamol may be administered as a single dose administration of two swallow formulations each containing 500 mg paracetamol, a water uptake agent and pH modulating agent in an amount sufficient to neutralize from about 0.3 mL to about 55 mL 0.1 N hydrochloric acid and/or to neutralize from about 0.03 mmol to about 5.5 mmol of a monoprotic acid.

In one embodiment, the swallow formulation is intended for use by an infant and paracetamol may be in the range of 100 to 250 mg.

In another embodiment, the swallow formulation is intended for use by an adult and paracetamol may be in the range of 250 to 1000 mg.

As indicated above, the most preferred pH modulating agent is sodium bicarbonate and/or calcium carbonate and/or magnesium carbonate and/or potassium bicarbonate which in the swallow formulation is present in from about 25 mg to 450 mg per swallow formulation such as 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449 or 450 mg.

The swallow formulation may also contain additional pharmaceutically active agents for example other analgesics such as codeine, hydrocodone, oxycodone, tramadol and propoxyphene, anti-inflammatory analgesics such as aspirin and ibuprofen, decongestants such as pseudoephedrine and phenylephrine, antitussives such as pholcodine and dextromethorphan, expectorants such as guaifenesin and bromhexine, non-sedating and sedating antihistamines such as diphenhydramine and chlorpheniramine and muscle relaxants such as doxylamine. Formulations may also contain a pharmaceutically acceptable adjuvant such as caffeine.

Accordingly, in one preferred embodiment, the present invention provides a swallow formulation comprising paracetamol incorporated as a rapidly dissolving form of paracetamol as described herein, a water uptake agent and one or more pH modulating agents wherein the pH modulating agent in a dose of paracetamol is in an amount sufficient to neutralize from about 0.6 mL to about 110 mL 0.1 N hydrochloric acid and/or to neutralize from about 0.06 mmol to about 11 mmol of a monoprotic acid, and one or more additional pharmaceutically active ingredients wherein 70% of the paracetamol dissolves from the swallow formulation within 180 seconds in USP dissolution apparatus 2 with 900 mL 0.05 N hydrochloric acid at 30 rpm and 37° C.

Particularly preferred swallow formulations include swallow formulations comprising about 50-65% paracetamol, 21-26% pH modulating agent and about 12-18% water uptake agent by weight of swallow formulation and swallow formulations comprising 50-65% paracetamol, 21-26% sodium bicarbonate, 7-9% crospovidone and 5-7% starch derivative by weight of swallow formulation.

In another aspect of the present invention there is provided a dosage form comprising a swallow formulation comprising paracetamol incorporated as a rapidly dissolving form of paracetamol, a water uptake agent and one or more pH modulating agents wherein the pH modulating agent in a dose of paracetamol is in an amount sufficient to neutralize from about 0.6 mL to about 110 mL 0.1 N hydrochloric acid and/or to neutralize from about 0.06 mmol to about 11 mmol of a monoprotic acid, in combination with a second component, wherein the dissolution of paracetamol from the dosage form is different than the dissolution of paracetamol from the swallow formulation.

A “swallow formulation” is any formulation that is administered to a subject by the action of swallowing. A “dosage form” comprising the swallow formulation can be a coated tablet or capsule which does not have the same dissolution characteristics as the swallow formulation itself.

The dosage form may be a tablet, capsule, powder, cachet, paste, colloid, gel or melt. The dose form may optionally be in a chewable form.

The dosage form of the present invention may be coated, uncoated and/or layered tablet. Suitable coatings include water soluble polymer based coatings such as, povidone or hypromellose. Suitable coating polymers may also be a derivative of cellulose (cellulose acetophthalate, hypromellose phthalate) or a derivative of an acrylic polymer (methacrylate acid copolymer). Optionally, the dosage form may be coated with gelatin.

The dosage form may contain one or more further pharmaceutically active agents.

In one embodiment, the dosage form is a multi phase release dosage form containing further paracetamol having a dissolution of less than 30% in 180 seconds in USP dissolution apparatus 2 with 900 mL 0.05 N hydrochloric acid at 30 rpm and 37° C.

Swallow formulations of the present invention may be manufactured by admixing the ingredients simultaneously or sequentially and then converting into a dosage unit such as a tablet, capsule, wafer or the like.

Tablets of the present invention may be manufactured by direct compression or granulation and compression for example.

The present invention further contemplates a method for the amelioration of the symptoms of pain, fever or discomfort in a subject, said method comprising administering to said subject a swallow formulation comprising paracetamol incorporated as a rapidly dissolving form of paracetamol as described herein, a water uptake agent and one or more pH modulating agents wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, the administration being for a time and under conditions to prevent or ameliorate symptoms of the condition.

Another aspect of the present invention contemplates a method for effecting pain and/or fever management in a subject experiencing pain and/or fever or anticipating to experience pain and/or fever, said method comprising administering to said subject a swallow formulation comprising paracetamol incorporated as a rapidly dissolving form of paracetamol as described herein, a water uptake agent and one or more pH modulating agents wherein the pH modulating agent is in an amount sufficient to neutralize from about 0.6 mL to about 110 mL 0.1 N hydrochloric acid and/or to neutralize from about 0.06 mmol to about 11 mmol of a monoprotic acid, the administration being for a time and under conditions to prevent or ameliorate symptoms of the condition.

These methods may also involve the swallow formulation having one or more pharmaceutically acceptable excipients.

The present invention is further described by the following non-limiting Examples.

### EXAMPLES

### Example 1

### Selection of pH Modulating Agents Based on Pharmacokinetic Studies

This non-limiting Example includes a range of different formulations based on rapidly dissolving paracetamol with carbonates as a pH modulating agent over the range 100-800 mg per dose as covered by this invention and the presence of water uptake agents that demonstrate improved tablet dissolution and AUC20 values. Formulation 1 covers the formulation and preparation of paracetamol-PVP crystals which were used as the rapidly dissolving paracetamol in many of the formulations.

All formulations were tested in fasted healthy human subjects, administering two tablets equivalent to a 1000 mg dose of paracetamol with water. Plasma levels of paracetamol were measured for up to 8 hours post-dosing, with at least 10 samples taken during the first hour. In vitro tablet dissolution was measured using USP dissolution apparatus 2 with 900 mL 0.05N hydrochloric acid at 30 rpm and 37° C.

A linear in vitro-in vivo correlation (IVIVC) (R²=0.60) was established between paracetamol AUC20 and the fitted tablet dissolution exponential rate constant (k_{diss}) based on the evaluation of 23 different tablet formulations with each formulation evaluated in 5 subjects in a crossover design. Accordingly, an AUC20 of approximately 150 min.mg.L⁻¹ corresponds to about 70% dissolution of paracetamol in 180 sec. A poorer linear correlation existed between Tₘₐₓ and k_{diss} for the 23 formulations (R²=0.29). A low Tₘₐₓ is generally associated with a high AUC20 but it was found that Tₘₐₓ begins to plateau at an AUC20 of approximately 120 min.mg.L⁻¹, supporting the use of AUC20 rather than Tₘₐₓ as the preferred in vivo measure for rate of absorption.

A validation crossover study of two test and two commercially available formulations in 26 healthy fasted subjects gave a linear correlation between the mean AUC20 and in vitro dissolution rate constant, k_{diss} (R²=0.990). This regression suggests that an AUC20 of 150 min.mg.L⁻¹ corresponds to about 70% of paracetamol dissolved from the formulations in 180 sec. The linear correlation between AUC20 and the % paracetamol dissolved at 180 seconds was R²=0.996.

The following tables provide examples of some of the formulations with in vivo pharmacokinetic data and in vitro dissolution performance.

TABLE 1
Formulation 1 For A Rapidly Dissolving Paracetamol

| No | Ingredient | mg/tablet |
|---|---|---|
| 1 | Povidone K-30 (PVP) | 20-45 |
| 2 | Water | |
| 3 | 96% v/v ethanol | |
| 4 | Paracetamol | 500 |

Procedure
A. Prepare a solution of approximately 1% w/w of 1 in 2 in a vessel and cool to 2-5°C.
B. Heat 3 to around 75°+C., and add sufficient paracetamol to prepare a solution of approximately 35% w/w.
C. Add B to A in the ratio approximately 1 to 3 by weight
D. Stir for 5 seconds leaving the mixing vessel on ice.
E. After 20 minutes, filter the resultant crystals under vacuum and dry at 40°C. to a moisture content of approximately 1%.
F. Screen through an appropriate sieve (˜840 μm).
G. Assay for paracetamol content.

The quantities of solvents, stirring procedures and settling time prior to filtration need to be determined for each batch size. Different ratios of ethanol and water can be used as solvents for the two phases.

Typically the crystals have a volume median diameter (D₅₀) below 300 μm and perform as a rapidly dissolving form of paracetamol.

The resultant crystals typically contain 4-8% PVP, and are assayed for paracetamol content before use. This allows the quantity equivalent to 500 mg paracetamol to be calculated for further processing. The crystals are blended with other ingredients in any formulation before conversion to the finished product, such as by compression to produce a tablet or by filling the powder blend into capsules.

Formulations 2-10 are some of those that were tested in the multiple 5 subject crossover studies to demonstrate the effect of pH modulating agents on the in vitro dissolution and in vivo pharmacokinetic parameters. All are direct compression formulations prepared by blending the ingredients prior to compression.

TABLE 2
Formulations 2-10 Containing Rapidly Dissolving Paracetamol

| Ingredients | Rapidly Dissolving Paracetamol | | | | | | | | |
|---|---|---|---|---|---|---|---|---|---|
| pH | Carbonates including 50-400 mg sodium | | | | | | | | |
| modulating | bicarbonate | | | | | | | Non | |
| agent | (100-800 mg per dose) | | | | | | | carbonates | None |
| Formulation Number | 2 | 3 | 4 | 5 | 6 | 7 | 8 | 9 | 10 |
| Crystals from | 538 | 538 | 526 | 543 | 0 | 538 | 526 | 526 | 526 |
| Formulation 1 | | | | | | | | | |
| equivalent to 500 mg | | | | | | | | | |
| paracetamol | | | | | | | | | |
| Paracetamol, | 0 | 0 | 0 | 0 | 500 | 0 | 0 | 0 | 0 |
| micronised | | | | | | | | | |
| Sodium | 50 | 200 | 200 | 200 | 200 | 400 | 125 | 0 | 0 |
| bicarbonate | | | | | | | | | |
| Potassium | 0 | 0 | 0 | 0 | 0 | 0 | 100 | 0 | 0 |
| bicarbonate | | | | | | | | | |
| Citric acid | 6 | 0 | 25 | 25 | 25 | 50 | 0 | 0 | 0 |
| monohydrate | | | | | | | | | |
| Monohydrogen | 0 | 0 | 0 | 0 | 0 | 0 | 0 | 200 | 0 |
| phosphate | | | | | | | | | |
| Urea | 0 | 0 | 0 | 0 | 0 | 0 | 0 | 30 | 0 |
| Mannitol | 0 | 0 | 0 | 50 | 50 | 0 | 0 | 0 | 0 |
| Starch 1500 | 50 | 50 | 50 | 0 | 0 | 50 | 50 | 50 | 50 |
| Povidone K-30 | 0 | 0 | 0 | 0 | 15 | 0 | 0 | 0 | 0 |
| Crospovidone | 60 | 60 | 60 | 60 | 60 | 60 | 60 | 60 | 60 |
| Stearic acid | 7 | 9 | 9 | 8 | 8 | 11 | 9 | 9 | 6 |

TABLE 3
In Vitro Dissolution and In Vivo Data for Formulations 2-10

| | Carbonates including 50-400 mg | | |
|---|---|---|---|
| | sodium bicarbonate | Non | |
| pH modulating agent | (100-800 mg per dose) | carbonates | None |
| Mean AUC20 | 255 | 89 | 144 |
| min · mg · L⁻¹ | | | |
| (pooled data) | | | |

Formulation Number

| | 2 | 3 | 4 | 5 | 6 | 7 | 8 | 9 | 10 |
|---|---|---|---|---|---|---|---|---|---|
| % released | 92 | 91 | 91 | 96 | 84 | 79 | 75 | 9 | 20 |
| in 90 sec | | | | | | | | | |
| Mean AUC20 min · mg · L⁻¹ | 241 | 238 | 256 | 296 | 205 | 309 | 201 | 89 | 144 |
| Mean Tₘₐₓ min | 14.4 | 13.3 | 18.3 | 14.4 | 17.7 | 14.9 | 19.5 | 30.2 | 39.6 |

Formulations 11 and 12 are those containing rapidly dissolving paracetamol with pH modulating agent that were evaluated in vivo in 26 healthy fasted subjects compared with two commercially available brands of paracetamol described as “rapid release products”.

TABLE 4
Formulation 11 Prepared By Granulation

| No | Ingredient | mg/tablet |
|---|---|---|
| Part 1 | | |
| 1 | Povidone K-29/32 | 20 |
| 2 | Water | |
| 3 | Crospovidone | 10 |
| 4 | Paracetamol, micronised | 500 |
| Part 2 | | |
| 5 | Sodium bicarbonate | 200 |
| 6 | Starch 1500 | 50 |
| 7 | Crospovidone | 60 |
| 8 | Stearic acid | 8 |

Procedure
Part 1
A. Prepare 13% w/w solution of 1 in 2.
B. Blend 3 and 4.
C. Spray A onto B in a granulator or mixer to produce a granule suitable for compression.
D. Dry at 40°C. to a moisture content ˜1%.
E. Screen through a 1,410 μm sieve.
   Part 2
F. Screen 5, 6 and 7 through a 250 μm sieve.
G. Blend Part 1 with F.
H. Screen 8 through a 250 μm sieve.
I. Blend H with G.

J. Compress.

TABLE 5
Formulation 12 Prepared By Direct Compression

| Item | | |
|---|---|---|
| number | Ingredient | mg/tablet |
| 1 | Crystals from Formulation 1 equivalent | 519 |
| | to 500 mg Paracetamol | |
| 2 | Sodium bicarbonate | 200 |
| 3 | Starch 1500 | 50 |
| 4 | Crospovidone | 60 |
| 5 | Stearic acid | 8 |

Procedure
A. Blend 1, 2, 3 and 4
B. Blend 5 with A

C. Compress.

TABLE 6
In Vitro And In Vivo Data For Formulations 11 And 12 Compared
With Two Commercially Available Paracetamol Products A And B

| | % dissolved in | |
|---|---|---|
| Product | 90 seconds | AUC20 min · mg · L⁻¹ |
| 11 | 81 | 197 |
| 12 | 84 | 207 |
| Commercial Product A | 5 | 80 |
| Commercial Product B | 1 | 100 |

### Example 2

### Selection of Rapidly Dissolving Paracetamol

The following data demonstrate the effect of the addition of a pH modulating agent, sodium bicarbonate, on the in vitro dissolution of tablets made with different grades of paracetamol. Six different grades of paracetamol were tested in Formulations 13-18. For each sample of paracetamol, two formulations were prepared; one without pH modulating agent designated A, and one with pH modulating agent designated B.

TABLE 7
Formulations A and B

| Item | | mg/tablet | |
|---|---|---|---|
| number | Ingredient | Formulation A | Formulation B |
| 1 | Paracetamol | 100 | 100 |
| 2 | Sodium bicarbonate | 0 | 100 |
| 3 | Microcrystalline cellulose | 270 | 270 |
| 4 | Crospovidone | 25 | 25 |
| 5 | Magnesium stearate | 5 | 5 |

Procedure
A. Blend 1, 2, 3 and 4
B. Blend 5 with A
Compress.

In vitro dissolution was measured using USP dissolution apparatus 2 with 900 mL 0.05N hydrochloric acid at 30 rpm and 37° C. The % paracetamol dissolved at 30 seconds was compared for each grade of paracetamol with and without the presence of a pH modulating agent, sodium bicarbonate. The % increase in dissolution caused by the sodium bicarbonate was calculated.

It was found that the increase in dissolution caused by sodium bicarbonate is much higher for certain grades of paracetamol. For the purposes of this invention, where the increase is greater than 500%, these grades of paracetamol are described as rapidly dissolving paracetamol. Paracetamol used in Formulations 13-16 meet the definition of rapidly dissolving paracetamol and those used in Formulations 17-18 do not.

TABLE 8
The Effect Of Paracetamol Grade On Dissolution Of Tablets Formulated With
And Without Sodium Bicarbonate

| | | | % paracetamol dissolved | | |
|---|---|---|---|---|---|
| Paracetamol | Volume | | after 30 sec | | % Increase |
| Sample | Median | Surface Area by | Without | With 100 mg | with Sodium |
| Formulation | Diameter | argon | Sodium | Sodium | Bicarbonate |
| Number | (D₅₀) μm | m² · g⁻¹ | Bicarbonate A | Bicarbonate B | B/A × 100 |
| 13 | 17 | 0.661 | 4.71 | 45.26 | 961 |
| 14 | 65 | 0.260 | 1.83 | 21.01 | 1148 |
| 15 | 203 | 0.365 | 2.36 | 32.84 | 1392 |
| 16 | 232 | 0.091 | 1.76 | 17.28 | 982 |
| 17 | 398 | 0.063 | 0.46 | 0.72 | 157 |
| 18 | 412 | 0.057 | 1.19 | 0.92 | 77 |

### Example 3

### Selection of Range of Water Uptake Agents Based on In Vitro Dissolution Studies

Various excipients used in the formulations can be generally classified as water uptake agents in addition to their specific categorization as disintegrants, wicking agents, binders and fillers. Microcrystalline cellulose is an example that can be described in all these categories. For the purposes of this invention, any excipients which are described in any of these categories are considered under the definition of water uptake agent.

It was found that in vitro dissolution did not occur rapidly unless there was sufficient water uptake agent present in combination with the pH modulating agent. It was further noted that a reduction in the in vitro dissolution of tablets was associated with an increase in the level of pH modulating agent, and that the dissolution performance could be restored by increasing the level of water uptake agent. Formulation 10 which contains water uptake agents but without any pH modulating agent shows slow dissolution of paracetamol reaching only 20% in 180 seconds. This compares with dissolution above 70% in 90 seconds when sufficient amounts of both agents are present in the formulation.

The % of water uptake agents in the tablet and the ratio of the water uptake agents to the weight of pH modulating agents are important to achieve the target in vitro dissolution performance of the resultant tablet.

TABLE 9
Formulations With Dissolution Greater Than 70% In 180 Seconds
Containing Different Water Uptake Agents

| | | mg/tablet | | | | |
|---|---|---|---|---|---|---|
| Item | | Formulation Number | | | | |
| No | Ingredient | 19 | 20 | 21 | 22 | 23 |
| 1 | Paracetamol, micronised | 500 | 500 | 500 | 100 | 100 |
| 2 | Sodium bicarbonate | 275 | 275 | 275 | 100 | 40 |
| 3 | Microcrystalline cellulose | 0 | 0 | 0 | 270 | 0 |
| 4 | Povidone K-30 | 0 | 0 | 0 | 0 | 4 |
| 5 | Starch 1500 | 70 | 70 | 70 | 0 | 10 |
| 6 | Croscarmellose | 0 | 67 | 0 | 0 | 0 |
| 7 | Sodium starch glycolate | 0 | 0 | 67 | 0 | 0 |
| 8 | Crospovidone | 67 | 0 | 0 | 0 | 12 |
| 9 | Stearic acid | 7 | 7 | 7 | 0 | 0 |
| 10 | Magnesium stearate | 0 | 0 | 0 | 5 | 1 |

Procedure
A. Blend items 1-8 according to the specific formulation
B. Blend item 9 or 10 with A according to the specific formulation
C. Compress.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ameer et al., J. Pharm. Sci, 72:955-958, 1983
Chen et al, Clin. Pharmacokinet. 40(8):565-72, 2001
Grattan et al., Eur. J. Pharm. Biopharm. 49(3):225-229, 2000
FDA Guidance for Industry, Bioavailability & Bioequivalence Studies for Orally Administered Drug Products—General Considerations, March 2003
Luthy, et al. Schweiz Med. Wochenschr 123 (Suppl 50)/II:406, 1993
McNeil Consumer & Speciality Pharmaceuticals, Submission to the FDA on acetaminophen for 19 Sep. 2002 Non-prescription Drugs Advisory Committee Meeting.
Nielsen et al. Eur. J. Clin. Pharmacol. 42(3): 261-264, 1992
Prescott, Medical Clinics of North America, 58:907-916, 1974
Prescott Am. J. Ther. 7(2):143-147 2000
Rumble et al., Clin. Pharmacokinet. 20 (2):167-173, 1991
Rumack et al, Pediatrics 62(Suppl):898-903, 1978
U.S. Patent Application No. 20040170681
U.S. Patent Application No. 20040204475
U.S. Pat. No. 6,316,025

1. A swallow formulation comprising
(i) paracetamol having a volume median diameter (D₅₀) of less than about 350 μm and a surface area of greater than about 0.07 m².g⁻¹;
(ii) a pH modulating agent in an amount sufficient to neutralize from about 0.6 mL to about 110 mL 0.1 N hydrochloric acid; and
(iii) an agent that facilitates water uptake into the formulation, wherein at least 70% of the paracetamol is dissolved from the swallow formulation within 180 seconds in USP dissolution apparatus 2 with 900 mL 0.05 N hydrochloric acid at 30 rpm and 37° C.

2. The swallow formulation of claim 1, wherein at least 80% of the paracetamol is dissolved from the swallow formulation within 180 seconds in USP dissolution apparatus 2 with 900 mL 0.05 N hydrochloric acid at 30 rpm and 37° C.

3. The swallow formulation of claim 1, wherein the swallow formulation is a tablet and at least 70% of the paracetamol is dissolved from the swallow formulation within 120 seconds in USP dissolution apparatus 2 with 900 mL 0.05 N hydrochloric acid at 30 rpm and 37° C.

4. The swallow formulation of claim 3, wherein at least 80% of the paracetamol is dissolved from the swallow formulation within 120 seconds in USP dissolution apparatus 2 with 900 mL 0.05 N hydrochloric acid at 30 rpm and 37° C.

5. The swallow formulation of claim 3, wherein at least 70% of the paracetamol is dissolved from the swallow formulation within 90 seconds in USP dissolution apparatus 2 with 900 mL 0.05 N hydrochloric acid at 30 rpm and 37° C.

6. The swallow formulation of claim 5, wherein at least 80% of the paracetamol is dissolved from the swallow formulation within 90 seconds in USP dissolution apparatus 2 with 900 mL 0.05 N hydrochloric acid at 30 rpm and 37° C.

7. The swallow formulation of claim 1, wherein a single dose administration of 1000 mg paracetamol with water to a fasted healthy human subject provides a mean AUC20 of more than about 150 min.mg.L⁻¹.

8. The swallow formulation of claim 1, wherein a single dose administration of 1000 mg paracetamol with water to a fasted healthy human subject provides a mean AUC20 of more than about 170 min.mg.L⁻¹.

9. The swallow formulation of claim 1, wherein the paracetamol used in the formulation exhibits a dissolution rate in USP dissolution apparatus 2 using 900 mL of 0.05 N hydrochloric acid at 30 rpm and 37° C. of at least 30% in 180 seconds.

10. The swallow formulation of claim 1, wherein the pH modulating agent is soluble or dispersible.

11. The swallow formulation of claim 10, wherein the at least one pH modulating agent is a base.

12. The swallow formulation of claim 11, wherein the at least one pH modulating agent is a carbonate.

13. The swallow formulation of claim 12, wherein the carbonate is selected from the group consisting of sodium bicarbonate, potassium bicarbonate, calcium carbonate, magnesium carbonate, sodium carbonate, ammonium carbonate, disodium glycine carbonate, sodium glycine carbonate, lysine carbonate, and arginine carbonate.

14. The swallow formulation of claim 13, wherein the carbonate is water soluble.

15. The swallow formulation of claim 14, wherein the carbonate is a sodium carbonate.

16. The swallow formulation of claim 12, wherein the pH modulating agent is sodium bicarbonate in an amount between about 50 mg and 400 mg and the paracetamol is in an amount of about 500 mg.

17. The swallow formulation of claim 1, wherein the pH modulating agent is present in an amount ranging from about 2% to about 90% by weight of the swallow formulation.

18. The swallow formulation of claim 17, wherein the pH modulating agent is present in an amount ranging from about 2% to about 80% by weight of the swallow formulation.

19. The swallow formulation of claim 18, wherein the pH modulating agent is present in an amount ranging from about 2% to about 70% by weight of the swallow formulation.

20. The swallow formulation of claim 1, wherein the ratio of paracetamol to pH modulating agent ranges from about 0.05:1 to about 30:1.

21. The swallow formulation of claim 1, wherein the amount of rapidly dissolving paracetamol dissolved from the swallow formulation is at least about 3 times greater than the amount of rapidly dissolving paracetamol dissolved from a swallow formulation without a carbonate pH modulating agent after 30 seconds in USP dissolution apparatus 2 with 900 mL 0.05 N hydrochloric acid at 30 rpm and 37° C.

22. The swallow formulation of claim 1, wherein the paracetamol has a volume median diameter (D₅₀) of less than about 300 μm.

23. The swallow formulation of claim 1, wherein the paracetamol has a surface area to mass ratio of greater than about 0.08 m².g⁻¹.

24. The swallow formulation of claim 1, wherein the rapidly dissolving form of paracetamol is a salt, ester, amide, pro-drug, or derivative of paracetamol.

25. The swallow formulation of claim 1, wherein the rapidly dissolving form of paracetamol is the product of paracetamol crystallized in the presence of one or more crystallization modifiers.

26. The swallow formulation of claim 25, wherein the crystallization modifier is a polymer, protein, or mixture thereof.

27. The swallow formulation of claim 25, wherein the crystallization modifier is polyvinylpyrrolidone.

28. The swallow formulation of claim 1, wherein the rapidly dissolving form of paracetamol is in the form of granules.

29. The swallow formulation of claim 28, wherein the granules comprise at least one of the pH modulating agent or the water uptake agent.

30. The swallow formulation of claim 28, further comprising at least one of extra granular paracetamol, pH modulating agent, or water uptake agent.

31. The swallow formulation claims 28, wherein the granules further comprise a disintegrant.

32. The swallow formulation of claim 31, wherein the disintegrant is selected from the group consiting of crospovidone, croscarmellose, sodium starch glycolate, starch, and starch derivatives.

33. The swallow formulation of claim 1, wherein the water uptake agent is selected from the group consisting of cross-linked polyvinylpyrrolidone (crospovidone), croscarmellose sodium, sodium starch glycolate, povidone, starch, starch derivatives, low substituted hydroxypropylcellulose, hydroxypropylcellulose, alginic acid, sodium alginate, calcium sulfate, calcium carboxymethylcellulose, microcrystalline cellulose, powdered cellulose, colloidal silicon dioxide, docusate sodium, guar gum, magnesium aluminium silicate, methylcellulose, polacrilin potassium, silicified microcrystalline cellulose, magnesium oxide, tragacanth, mannitol, sorbitol, xylitol, sucrose, lactose, fructose, maltose, polyethylene glycol, aminoacids, cyclodextrin, urea and polyvinylpyrrolidone.

34. The swallow formulation of claim 1, wherein the water uptake agent is present in an amount ranging from about 2% to about 80% by weight of the swallow formulation.

35. The swallow formulation of claim 34, wherein the water uptake agent is present in an amount ranging from about 2% to about 60% by weight of the swallow formulation.

36. The swallow formulation of claim 1, wherein the swallow formulation comprises about 50-65% of paracetamol, 21-26% of pH modulating agent, and about 12-18% of water uptake agent by weight of the swallow formulation.

37. The swallow formulation of claim 1, comprising about 50-65% of paracetamol, about 21-26% of sodium bicarbonate, about 7-9% of crospovidone, and about 5-7% of a starch derivative by weight of swallow formulation.

38. The swallow formulation of claim 1, further comprising one or more other pharmaceutically active agents.

39. The swallow formulation of claim 1, wherein the swallow formulation comprises paracetamol in an amount ranging from 100 mg to 1000 mg, the pH modulating agent is present in an amount sufficient to neutralize from about 0.3 mL to about 55 mL 0.1N hydrochloric acid, and/the ratio of the pH modulating agent to the paracetamol ranges from 0.05:1 to 30:1 by weight.

40. The swallow formulation of claim 1, wherein the swallow formulation comprises 500 mg paracetamol and from about 25 to 450 mg sodium bicarbonate.

41. A dosage form comprising (i) the swallow formulation of claim 1 and (ii) a second component, wherein the amount of paracetamol dissolved from the dosage form in USP dissolution apparatus 2 with 900 mL 0.05 N hydrochloric acid at 30 rpm and 37° C. is different from the amount of paracetamol dissolved from the swallow formulation in USP dissolution apparatus 2 with 900 mL 0.05 N hydrochloric acid at 30 rpm and 37° C.

42. The dosage form of claim 41, wherein the second component is a coating and the dosage form is a coated tablet.

43. The dosage form of claim 41, wherein the second component is a capsule and the swallow formulation is in the capsule.

44. The dosage form of claim 41, wherein the second component comprises additional paracetamol and provides sustained release of the additional paracetamol.

45. The dosage form of claim 44, wherein is the additional paracetamol has a dissolution in USP dissolution apparatus 2 using 900 mL of 0.05M HCl with the paddle spinning at 30 rpm at 37° C. of less than 30% in 180 seconds.

46. A method for treating the symptoms of pain, fever, or discomfort in a subject comprising administering to said subject the swallow formulation of claim 1.

47. The method of claim 46, wherein the subject is a human.
